(19) United States
(12) Patent Application Publication
(10) Pub. No.: US 2024/0024415 A1
(43) Pub. Date: Jan. 25, 2024

(54) TREATMENT OF LOWER LIMB SPASTICITY

(71) Applicant: IPSEN BIOPHARM LIMITED, Wrexham (GB)

(72) Inventors: Donald PEARL, Basking Ridge, NJ (US); Ayanna SANTOS, Basking Ridge, NJ (US)

(73) Assignee: IPSEN BIOPHARM LIMITED, Wrexham (GB)

(21) Appl. No.: 18/168,713

(22) Filed: Feb. 14, 2023

Related U.S. Application Data
(63) Continuation of application No. 17/304,182, filed on Jun. 16, 2021, now abandoned.
Application No. 17/304,182 is a division of application No. 16/040,934, filed on Jul. 20, 2018, now abandoned.
(60) Provisional application No. 62/537,567, filed on Jul. 27, 2017.

(51) Int. Cl. A61K 38/16; G06G 1/00; A61K 38/48; G16H 20/17; A61P 21/00

(52) U.S. Cl. CPC A61K 38/164; G06G 1/001; A61K 38/4893; G16H 20/17; A61P 21/00

(57) ABSTRACT

A method of treating lower limb spasticity in a subject, particularly one which is 18 years of age or under, comprising administering to the subject an effective amount of botulinum neurotoxin. A composition for use in treating lower limb spasticity in a patient comprising botulinum neurotoxin. A tool for use in determining the dosage amount of an active agent to be administered to a subject.

The present invention relates to a method of treating lower limb spasticity in a subject, particularly one which is 18 years of age or under, for example between 2 and 17 years of age, comprising administering to the subject an effective amount of botulinum neurotoxin. The invention also relates also to a composition for use in treating lower limb spasticity in a patient comprising botulinum neurotoxin. In addition, the invention relates to a tool for use in determining the dosage amount of an active agent to be administered to a subject.

### BACKGROUND OF THE INVENTION

Spasticity is a condition in which there is an abnormal increase in muscle tone or stiffness in one or more muscles, which might interfere with movement thereof. Spasticity is typically caused by damage to nerve pathways in the brain or spinal cord that control muscle movement, and may occur in association with cerebral palsy, spinal cord injury, multiple sclerosis, stroke, and brain or head trauma.

Lower limb spasticity commonly involves spasticity in the gastrocnemius and soleus muscle complex located in the calf. These calf muscles are the chief extensors of the foot at the ankle-joint. In walking, they work to raise the heel from the ground.

Symptoms of spasticity may include increased muscle tone, rapid muscle contractions, exaggerated deep tendon reflexes, and/or muscle spasms. The degree of spasticity can vary from mild muscle stiffness to severe, painful, and uncontrollable muscle spasms.

It is known to treat spasticity in adults using botulinum neurotoxin. Applicant has found that botulinum neurotoxin may be used to treat lower limb spasticity in subjects 18 years of age or under, for example subjects between 2 and 17 years of age.

Botulinum neurotoxin is produced by Clostridium botulinum in the form of a large protein complex, consisting of botulinum neurotoxin itself complexed to a number of accessory proteins. There are at present seven different classes of botulinum neurotoxin, namely: botulinum neurotoxin serotypes A, B, C, D, E, F and G, all of which share similar structures and modes of action. Different BoNT serotypes can be distinguished based on inactivation by specific neutralizing anti-sera, with such classification by serotype correlating with percentage sequence identity at the amino acid level. Botulinum neurotoxin proteins of a given serotype are further divided into different subtypes on the basis of amino acid percentage sequence identity.

Botulinum toxin type A is mainly commercially available from Ipsen (DYSPORT®, Ipsen Limited, Slough, UK), Allergan (BOTOX®, Allergan Inc., Irvine, CA, USA), Merz Pharma (XEOMIN®, Merz Pharma GmbH, Frankfurt am Main, Germany), and Medytox (CORETOX®, Medytox Inc., Seoul, South Korea), whereas botulinum toxin type B is sold by Elan (MYOBLOC®/NEUROBLOC®, Solstice Neurosciences Inc., San Diego, CA, USA).

### SUMMARY OF THE INVENTION

The present invention relates in part to a method of treating lower limb spasticity in a subject, particularly one which is 18 years of age or under, for example between 2 and 17 years of age, comprising administering to the subject an effective amount of botulinum neurotoxin.

In an embodiment, the botulinum neurotoxin is of types A, B, C, D, E, F, and/or G.

In a particular embodiment, the botulinum neurotoxin is of type A.

In an embodiment, the botulinum neurotoxin is administered unilaterally. In a particular embodiment, botulinum neurotoxin is administered to an individual leg in an amount of 10 to 15 units per kilogram of the total body weight of the subject.

In an embodiment, the botulinum neurotoxin is administered bilaterally. In a particular embodiment, each leg of the subject is individually administered with 10 to 15 units per kilogram of the total body weight of the subject.

In an embodiment, the total amount of botulinum neurotoxin administered to the subject per treatment session is 1000 units or less.

In an embodiment, botulinum neurotoxin is administered at more than one site in any single muscle.

In an embodiment, no more than 0.5 mL of DYSPORT® is administered at any single site.

In an embodiment, a subsequent treatment session does not take place within 12 weeks of another treatment session. In a particular embodiment, a subsequent treatment session takes place 16 to 22 weeks after another treatment session.

In an embodiment, the botulinum neurotoxin is administered to the gastrocnemius-soleus complex. In a particular embodiment, botulinum neurotoxin is administered unilaterally in an amount of 10 to 15 units per kilogram of the total body weight of the subject. In another particular embodiment, botulinum neurotoxin is administered bilaterally in an amount of 20 to 30 units per kilogram of the total body weight of the subject.

In an embodiment, the botulinum neurotoxin is administered to a gastrocnemius of the subject. In a particular embodiment, botulinum neurotoxin is administered to an individual gastrocnemius in an amount of 6 to 9 units per kilogram of the total body weight of the subject. In certain embodiments, each gastrocnemius of the subject is administered with such an amount.

In an embodiment, the botulinum neurotoxin is administered to a soleus of the subject. In a particular embodiment, botulinum neurotoxin is administered to an individual soleus in an amount of 4 to 6 units per kilogram of the total body weight of the subject. In certain embodiments, each soleus of the subject is administered with such an amount.

In an embodiment, the botulinum neurotoxin is administered by injection to a muscle. In a particular embodiment, up to 4 injections are made to a gastrocnemius. In another particular embodiment, up to 2 injections are made to a soleus. In an embodiment injection sites are determined by palpation or by the use of injection guiding techniques such as electromyography or electrical stimulation.

In an embodiment, botulinum neurotoxin is administered in a dosage amount determined based in part on the weight of the subject, whether administration is to be unilateral or bilateral, whether administration will be to the gastrocnemius and/or the soleus, the severity of the spasticity, what muscles are affected by the spasticity, the presence of local muscle weakness, and/or the subject's history with treatment with botulinum neurotoxin.

In a particular embodiment, the method comprises the steps of: (A) obtaining the weight of the subject; (B) choosing unilateral or bilateral administration; (C) based on the weight of the patient and whether administration is to be unilateral or bilateral, determining the amount of botulinum neurotoxin to administer to the subject; and (D) administering botulinum neurotoxin in such an amount to a subject in need of such treatment.

The present invention also relates in part to a tool for use in determining the amount of active agent, for example botulinum neurotoxin, to administer to a subject.

In an embodiment, the tool comprises:
(A) a first member having thereon a first row of numbers indicative of the weight of the subject and a second row of numbers indicative of a dosage amount of the active agent to be administered to the subject, wherein the numbers in the first row indicating a specific weight are in latitudinal or radial alignment with corresponding numbers in the second row indicating a specific dosage amount to be administered to a subject having that specific weight; and
(B) a second member;
   wherein the first and second members are in a relationship with one another such that one member may be moved by a user to provide an indication as to the dosage amount of the active agent to be administered to a subject having a particular weight.

In an embodiment, both first and second members are planar surfaces that are in a longitudinal sliding arrangement with each other, with the second member overlaying the first member and having an opening or openings that allows for the rows on the first member to be viewed.

In another embodiment, the first member is a planar surface and the second member is a cursor in a longitudinal sliding arrangement with respect to the first member.

In another embodiment, the first member is a circular planar surface and the second member is a dial that pivots around the center point of the first member.

In an embodiment of the aforementioned method of treating lower limb spasticity in a subject, the dosage amount to be administered is determined using a tool as described above.

The invention further relates in part to a composition for use in the treatment of lower limb spasticity in a subject, particularly one which is 18 years of age or under, for example one which is between 2 and 17 years of age. The composition comprises an effective amount of botulinum neurotoxin.

In an embodiment, the composition comprises 10 to 15 units botulinum neurotoxin per kilogram of the total body weight of the subject for each leg to which botulinum neurotoxin is to be administered.

In an embodiment, the composition comprises an amount of botulinum neurotoxin to be administered to an individual gastrocnemius of the subject, the amount being 6 to 9 units per kilogram of the total body weight of the subject.

In an embodiment, the composition comprises an amount of botulinum neurotoxin to be administered to an individual soleus of the subject, the amount being 4 to 6 units per kilogram of the total body weight of the subject.

In an embodiment, the composition is in the form of a lyophilized powder.

In an embodiment, the composition is formed by reconstituting lyophilized powder comprising botulinum neurotoxin in a sodium chloride solution. In particular embodiments, the composition comprises 300 to 500 units of botulinum neurotoxin.

In an embodiment, the composition comprises botulinum neurotoxin and human serum albumin.

### DESCRIPTION OF THE DRAWINGS

FIG. 1 depicts an embodiment of the tool of the present invention. The tool comprises: (A) a first member (1) having thereon a first row of numbers (2) indicative of the weight of the subject, a second row of numbers (3) indicative of a dosage amount of the active agent to be administered to the subject having a specific weight, and additional rows of numbers (8) with one row indicating the dosage amount to be administered to the gastrocnemius of the subject's leg (9) and another row indicating the dosage mount to be administered to the soleus of the subject's leg (10); and (B) a second member (4). Both first and second members are planar surfaces and are in a relationship with one another such that one member may be moved by a user to provide an indication as to the dosage amount of the active agent to be administered to a subject having a particular weight. The second member overlays the first and contains an opening (5) that allows for the rows on the first member to be viewed. A first marking (11) on the second member is aligned with the row of numbers which indicate the weight of the subject (2), a second marking (12) on the second member is aligned with the row of numbers which indicate the dosage amount of the active agent to be administered to the subject (3), and third (13) and fourth markings (14) on the second member are aligned respectively with the row indicating the dosage amount of the active agent to be administered to the gastrocnemius of the subject's leg (9) and the row indicating the dosage amount of the active agent to be administered to the soleus of the subject's leg (10).

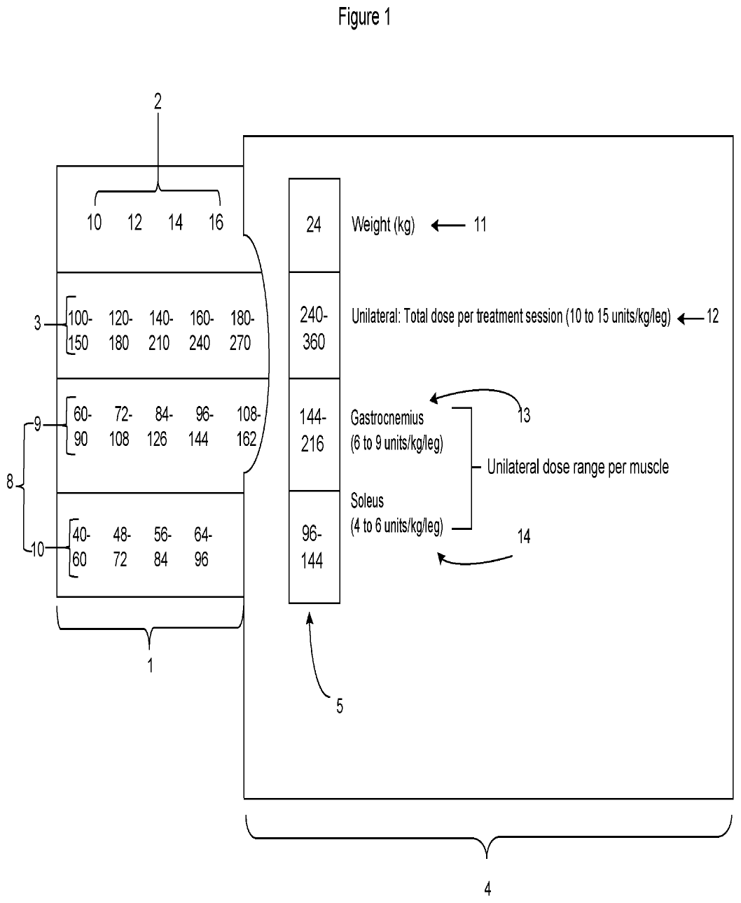

FIG. 2 depicts another embodiment of the tool of the present invention. The tool comprises: (A) a first member (1) having thereon a first row of numbers (2) indicative of the weight of the subject, a second row of numbers (3) indicative of a dosage amount of the active agent to be administered to the subject having a specific weight, and additional rows of numbers (8) with one row indicating the dosage amount to be administered to the gastrocnemius of the subject's leg (9) and another row indicating the dosage mount to be administered to the soleus of the subject's leg (10); and (B) a second member (4). Both first and second members are planar surfaces and are in a relationship with one another such that one member may be moved by a user to provide an indication as to the dosage amount of the active agent to be administered to a subject having a particular weight. The second member overlays the first and contains an openings (6) which each allow for one row on the first member to be viewed. A first marking (11) on the second member is aligned with the row of numbers which indicate the weight of the subject (2), a second marking (12) on the second member is aligned with the row of numbers which indicate the dosage amount of the active agent to be administered to the subject (3), and third (13) and fourth markings (14) on the second member are aligned respectively with the row indicating the dosage amount of the active agent to be administered to the gastrocnemius of the subject's leg (9) and the row indicating the dosage amount of the active agent to be administered to the soleus of the subject's leg (10).

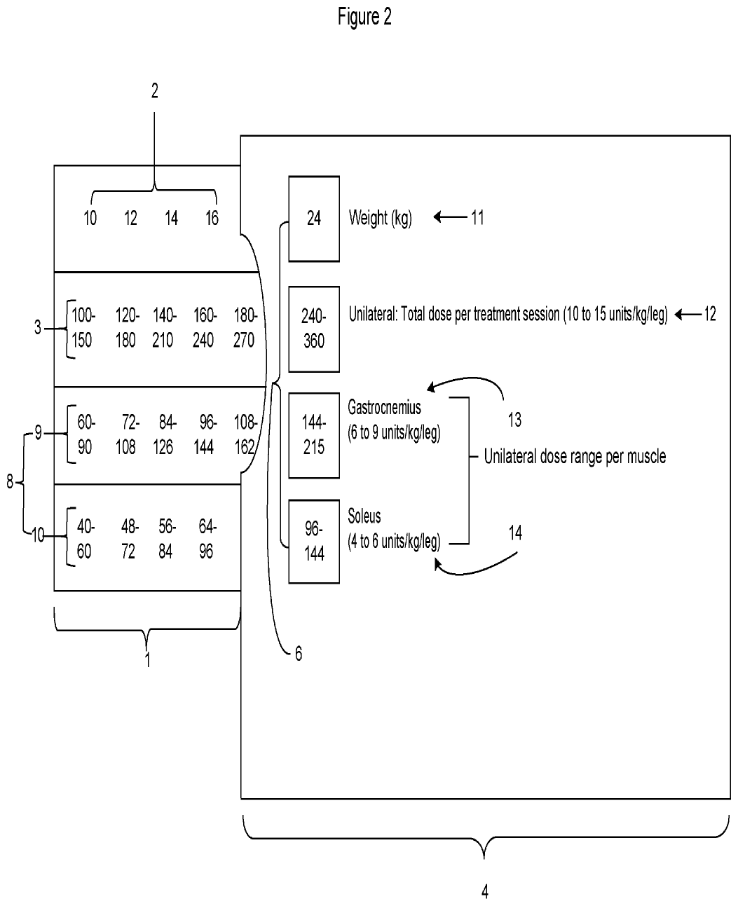

FIG. 3 depicts yet another embodiment of the tool of the present invention. The tool comprises: (A) a first member (1) having thereon a first row of numbers (2) indicative of the weight of the subject and a second row of numbers (3) indicative of a dosage amount of the active agent to be administered to the subject having a specific weight; and (B) a second member (4). The first member is a planar surface and the second member is a cursor. The second member is in a longitudinal sliding relationship with the first member such that it may be by a user to provide an indication as to the dosage amount of the active agent to be administered to a subject having a particular weight. The cursor is made of transparent material. A first marking (11) on the second member is aligned with the row of numbers which indicate the weight of the subject (2) and a second marking (12) on the second member is aligned with the row of numbers which indicate the dosage amount of the active agent to be administered to the subject (3). The numbers appear on a label (15). A line (7) is present on the cursor to provide an indication of the specific weight and dosage amount to be administered to a subject having that specific weight.

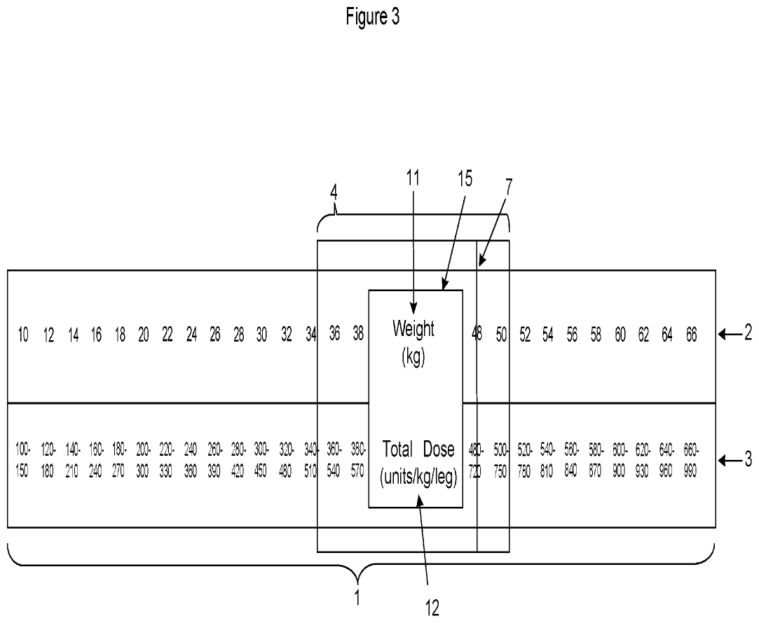

FIG. 4 depicts a further embodiment of the tool of the present invention. The tool comprises: (A) a first member (1) having thereon a first row of numbers (2) indicative of the weight of the subject and a second row of numbers (3) indicative of a dosage amount of the active agent to be administered to the subject having a specific weight; and (B) a second member (4). The first member is a planar surface and the second member is a cursor. The second member is in a longitudinal sliding relationship with the first member such that it may be by a user to provide an indication as to the dosage amount of the active agent to be administered to a subject having a particular weight. A first marking (11) on the second member is aligned with the row of numbers which indicate the weight of the subject (2) and a second marking (12) on the second member is aligned with the row of numbers which indicate the dosage amount of the active agent to be administered to the subject (3). The numbers appear on a label (15). The cursor contains an opening (5) that allows the user to view a specific weight amount and dosage amount to be administered to a subject having that specific weight.

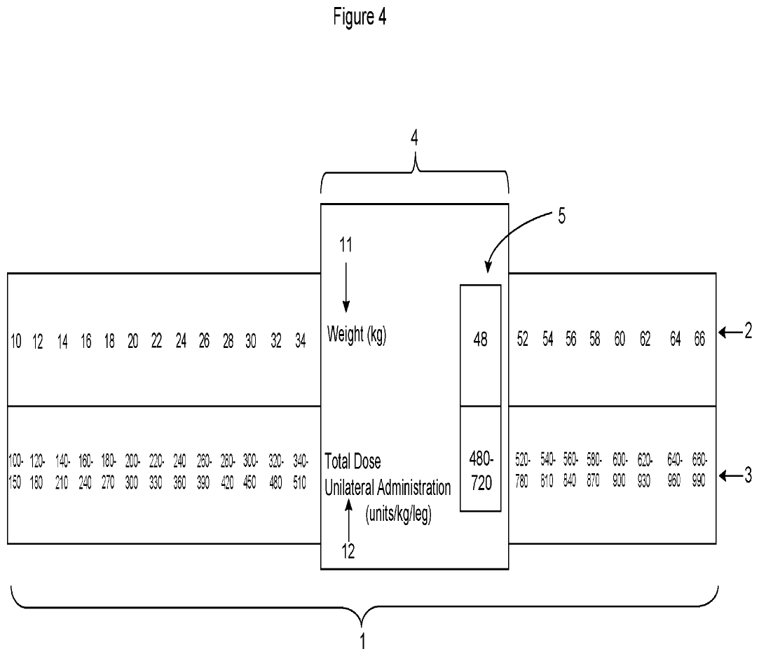

FIG. 5 depicts a yet further embodiment of the tool of the present invention. The tool comprises: (A) a first member (1) having thereon a first row of numbers (2) indicative of the weight of the subject and a second row of numbers (3) indicative of a dosage amount of the active agent to be administered to the subject having a specific weight; and (B) a second member (4). The first member is a circular planar surface and the second member is a dial that pivots around the center point of the first member such that it may be by a user to provide an indication as to the dosage amount of the active agent to be administered to a subject having a particular weight. The cursor contains an opening (5) that allows the user to view a specific weight amount and dosage amount to be administered to a subject having that specific weight. A first marking (11) on the second member is aligned with the row of numbers which indicate the weight of the subject (2) and a second marking (12) on the second member is aligned with the row of numbers which indicate the dosage amount of the active agent to be administered to the subject (3).

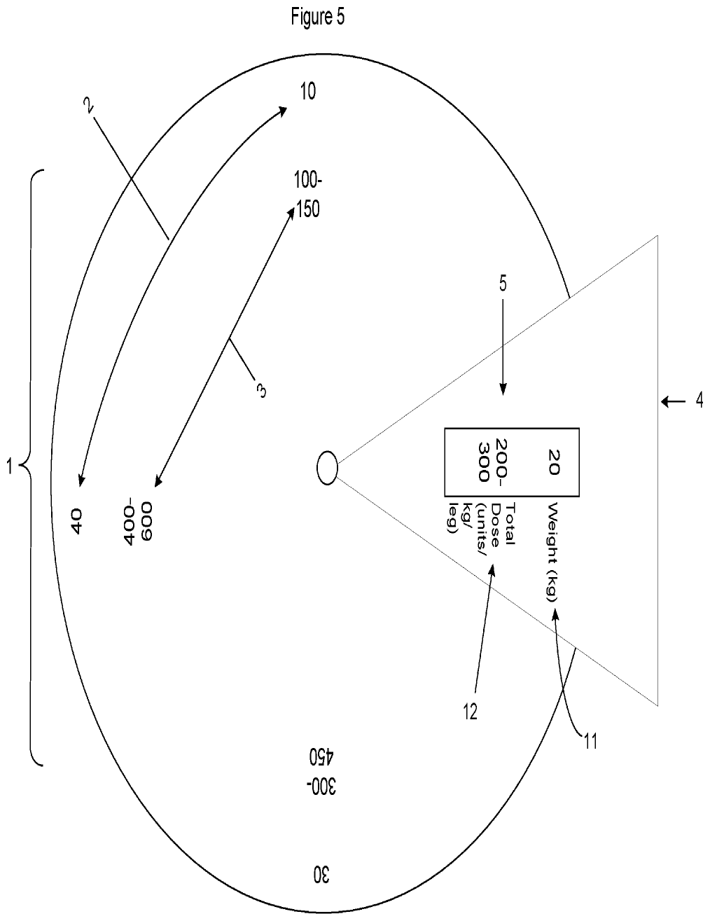

### DETAILED DESCRIPTION OF THE INVENTION

It is to be understood that the present invention is not limited to the embodiments described herein. Indeed, numerous variations, changes, and substitutions will now occur to those skilled in the art without departing from the invention. It should be understood that various alternatives to the embodiments of the invention described herein will be employed in practicing the invention.

In describing the invention, where a range of values is provided with respect to an embodiment, it is understood that each intervening value is encompassed within the embodiment.

The present invention relates in part to a method of treating lower limb spasticity in a subject, particularly one which is 18 years of age or under, for example one which is between 2 and 17 years of age, comprising administering to the subject an effective amount of botulinum neurotoxin. A “subject” as used herein refers to a mammal, in particular a human, in need of treatment for lower limb spasticity. For example, a subject may be a human patient in need of such treatment.

Botulinum neurotoxins are proteins produced by Clostridium botulinum. These proteins serve as neurotoxins. Upon selective uptake by endocytosis at nerve terminals, they block the release of synaptic vesicles. There are seven known serotypes of botulinum neurotoxin, specifically serotypes A, B, C, D, E, F, and G. As used herein, “botulinum neurotoxin” refers to naturally occurring botulinum neurotoxins, including in their high purity form, and recombinantly engineered botulinum neurotoxins, including those that contain modifications as compared to wild-type botulinum neurotoxins (e.g. in their amino acid sequence) so as to have similar or better properties than the wild-type botulinum neurotoxins. Botulinum toxin type A includes all types of botulinum toxin type A, including A1, A2 and A3. Botulinum toxin type C includes all types of botulinum toxin type C, including C1 or C2. The same applies mutatis mutandis to the other serotypes of toxins.

A “high purity botulinum neurotoxin” (whether of type A, B, C, D, E, F or G) refers to botulinum neurotoxin that is free from complexes with other proteins.

In an embodiment, the botulinum neurotoxin is of types A, B, C, D, E, F, and/or G or a recombinantly engineered botulinum neurotoxin having an amino acid sequence that has at least 50%, 60%, 70%, 80%, 90%, 95%, 97%, 98%, or 99% sequence identity to the amino acid sequence of a botulinum neurotoxin of types A, B, C, D, E, F, or G.

In an embodiment, the botulinum neurotoxin is of type A, also known as botulinum neurotoxin A. Botulinum neurotoxin A is, for example, sold as DYSPORT® by Ipsen Biopharmaceuticals, Inc. (Basking Ridge, New Jersey) and as BOTOX® by Allergan, Inc. (Parsippany-Troy Hills, New Jersey).

The botulinum neurotoxin may be administered unilaterally. The term “unilateral,” and its variants, when used herein in reference to administration refers to the administration of botulinum neurotoxin to one leg of the subject.

The potency of the toxin may be expressed as a multiple of the LD₅₀ value. One LD₅₀ unit is the equivalent amount of toxin which causes the death of 50% (one-half) of a group of test animals, such as laboratory mice. Alternative methods of determining the potency of the toxin may also be employed, including, for example, any method included in the European Pharmacopoeia monograph 01/2005:2113, which is hereby incorporated by reference.

In an embodiment, botulinum neurotoxin is administered unilaterally in an amount of 10 to 15 units per kilogram of the total body weight of the subject. As used herein, the term “unit” refers to a unit dose of DYSPORT®, which refers to the median intraperitoneal LD₅₀ dose of DYSPORT® in mice. It is noted that the median intraperitoneal LD₅₀ dose of DYSPORT® in mice is not necessarily the same as the median intraperitoneal LD₅₀ dose of another botulinum neurotoxin-containing product, such as BOTOX®, as different pharmaceutical preparations are produced differently. For example, units of BOTOX®, as described in the art, are not the same as the units of the present invention, which are units of DYSPORT®.

DYSPORT® is an injectable form of botulinum toxin type A (BoNT-A), which is isolated and purified from Clostridium bacteria producing BoNT-A. It is supplied as a lyophilized powder. DYSPORT® has approved therapeutic indications in the United States for the treatment of adults with Cervical Dystonia (CD), the treatment of Upper Limb Spasticity (ULS) in adult patients, and the treatment of lower limb spasticity in children to improve tone and spasticity. The medicine was first registered in the United Kingdom in 1990 for other uses and is licensed in more than 80 countries in eight different indications, with over 1,300 peer-reviewed publications.

Botulinum neurotoxin may also be administered bilaterally. The term “bilateral,” and its variants, when used herein in reference to administration refers to the administration of botulinum neurotoxin to both legs of the subject.

In an embodiment, botulinum neurotoxin is administered bilaterally to each leg of the subject in an amount of 10 to 15 units, 11 to 14 units, or 12 to 13 units per kilogram of the total body weight of the subject per leg.

In an embodiment, the total amount of botulinum neurotoxin administered to the subject per treatment session is 1000 units or less, 900 units or less, 800 units or less, 700 units or less, 600 units or less, or 500 units or less.

In an embodiment, subsequent treatment with botulinum neurotoxin following a previous such treatment does not take place within 12 weeks of the previous treatment. For example, the subsequent treatment may take place 16 to 22 weeks after the previous treatment.

The present invention contemplates the administration of botulinum neurotoxin may be administered by any means, for example parenterally, intra-muscularly, sub-cutaneously, intradermally, or transdermally. In an embodiment, administration may be by injection to a muscle.

Administration may, for example, be to the muscles of the gastrocnemius-soleus complex. For example, botulinum neurotoxin may be administered to a gastrocnemius of the subject. In an embodiment, botulinum neurotoxin is administered to an individual gastrocnemius in an amount of 6 to 9 units or 7 to 8 units per kilogram of the total body weight of the subject. In certain embodiments, each gastrocnemius of the subject is administered with such an amount. Botulinum may also or alternatively be administered to a soleus of the subject. In an embodiment, botulinum neurotoxin is administered to an individual soleus in an amount of 4 to 6 units or about 5 units per kilogram of the total body weight of the subject. In certain embodiments, each soleus of the subject is administered with such an amount.

Administration may also take place at more than one site in any single muscle. In an embodiment, no more than 0.5 mL, 0.25 mL, or 0.1 mL of DYSPORT® is administered at any single site.

The amount of botulinum neurotoxin to be administered may, for example be determined based in part on factors such as: the weight of the subject, whether administration is to be unilateral or bilateral, whether administration will be to the gastrocnemius and/or to the soleus, the severity of the subject's spasticity, what muscles are affected by the spasticity, the presence of local muscle weakness, and/or the subject's history with treatment with botulinum neurotoxin.

For example, when administration is unilateral, a total dosage of 10 to 15 units/kg of the total body weight of the subject may be administered. As such, 100 to 150 units may be administered to a patient having a total body weight of 10 kg, 200 to 300 units may be administered to a patient having a total body weight of 20 kg, 300 to 450 units may be administered to a patient having a total body weight of 30 kg, 400 to 600 units may be administered to a patient having a total body weight of 40 kg, 500 to 750 units may be administered to a patient having a total body weight of 50 kg, 600 to 900 units may be administered to a patient having a total body weight of 60 kg, and 700 to 1,050 units may be administered to a patient having a total body weight of 70 kg.

When administration is bilateral, for example, a total dosage of 20 to 30 units/kg of the total body weight of the subject may be administered. As such, 200 to 300 units may be administered to a patient having a total body weight of 10 kg, 400 to 600 units may be administered to a patient having a total body weight of 20 kg, 600 to 900 units may be administered to a patient having a total body weight of 30 kg, 800 to 1,200 units may be administered to a patient having a total body weight of 40 kg, 1,000 to 1,500 units may be administered to a patient having a total body weight of 50 kg, 1,200 to 1,800 units may be administered to a patient having a total body weight of 60 kg, and 1,400 to 2,100 units may be administered to a patient having a total body weight of 70 kg.

When a gastrocnemius of the subject is affected by spasticity, administration of botulinum neurotoxin may, for example, be to the gastrocnemius.

In embodiments wherein administration is unilateral, administration to the soleus may, for example, be 6 to 9 units/kg of the total body weight of the subject. For example, 60 to 90 units may be administered to a patient having a total body weight of 10 kg, 120 to 180 units may be administered to a patient having a total body weight of 20 kg, 180 to 270 units may be administered to a patient having a total body weight of 30 kg, 240 to 360 units may be administered to a patient having a total body weight of 40 kg, 300 to 450 units may be administered to a patient having a total body weight of 50 kg, 360 to 540 units may be administered to a patient having a total body weight of 60 kg, and 420 to 630 units may be administered to a patient having a total body weight of 70 kg. In embodiments wherein administration is bilateral, the above amounts may be individually administered to the gastrocnemius of each leg in the above amounts.

When a soleus of the subject is affected by spasticity, administration of botulinum neurotoxin may, for example, be to the soleus. In embodiments wherein administration is unilateral, administration to the soleus may, for example, be 4 to 6 units/kg of the total body weight of the subject. For example, 40 to 60 units may be administered to a patient having a total body weight of 10 kg, 80 to 120 units may be administered to a patient having a total body weight of 20 kg, 120 to 180 units may be administered to a patient having a total body weight of 30 kg, 160 to 240 units may be administered to a patient having a total body weight of 40 kg, 200 to 300 units may be administered to a patient having a total body weight of 50 kg, 240 to 360 units may be administered to a patient having a total body weight of 60 kg, and 280 to 420 units may be administered to a patient having a total body weight of 70 kg. In embodiments wherein administration is bilateral, the above amounts may be individually administered to the soleus of each leg in the above amounts.

The level of a patient's spasticity may, for example, be measured using the Modified Ashworth Scale (MAS) which measures the level of increase in resistance during passive soft-tissue stretching. A MAS score of 0 indicates no increase in muscle tone. A score of 1 indicates a slight increase in muscle tone, manifested by a catch and release or by minimal resistance at the end of the range of motion when the affected part(s) is moved in flexion or extension. A score of 1+ indicates a slight increase in muscle tone, manifested by a catch, followed by minimal resistance throughout the reminder (less than half) of the range of movement. A score of 2 indicates a more marked increase in muscle tone through most of the range of motion, but the affected part(s) easily moved. A score of 3 indicates considerable increase in muscle tone passive with movement being difficult. A score of 4 indicates that the affected part is rigid in flexion or extension.

In an embodiment, the amount of units administered to the subject may be directly proportional to the level of the subject's spasticity.

For example, when a subject is suffering from severe spasticity in his/her gastrocnemius, as indicated by a MAS score of 3 or above, administration may, for example, be 7.5 to 9 units/kg of the total body weight of the subject per leg (i.e. 7.5 to 9 units/kg for unilateral administration and 15 to 18 units/kg for bilateral administration.) In other embodiments, administration may be 8 to 9 units/kg per leg, 8.5 to 9 units/kg per leg, or 8.75 to 9 units/keg per leg.

For example, when a subject is suffering from severe spasticity in his/her soleus, as indicated by a MAS score of 3 or above, administration may, for example, be 5 to 6 units/kg of the total body weight of the subject per leg (i.e. 5 to 6 units/kg for unilateral administration and to 12 units/kg for bilateral administration.) In other embodiments, administration may be 5.5 to 6 units/kg per leg or 5.75 to 6 units/kg per leg.

By contrast, when a subject is suffering from less severe or minor spasticity in his/her gastrocnemius administration may, for example, be 6 to 7.5 units/kg of the total body weight of the subject per leg (i.e. 6 to 7.5 units/kg for unilateral administration and 12 to 15 units/kg for bilateral administration.) In other embodiments, administration may be 6 to 7 units/kg per leg, 6 to 6.5 units/kg per leg, or 6 to 6.25 units/keg per leg.

When a subject is suffering from less severe or minor spasticity in his/her soleus, administration may, for example, be 4 to 5 units/kg of the total body weight of the subject per leg (i.e. 4 to 5 units/kg for unilateral administration and 8 to 10 units/kg for bilateral administration.) In other embodiments, administration may be 4 to 4.5 units/kg per leg, or 4 to 4.25 units/keg per leg.

In addition, a lower dose may be administered if the subject has exhibited adverse effects following previous treatment with botulinum neurotoxin or exhibits local muscle weakness in the muscle to which the botulinum neurotoxin is to be administered. Examples of adverse effects include respiratory tract infection, nasopharyngitis, influenza, pharyngitis, cough, pyrexia, bronchitis, rhinitis, varicella, ear infection, gastroenteritis, vomiting, nausea, oropharyngeal pain, pain in extremities, muscular weakness, convulsion, and epilepsy. An example of such a lower dose is 10 to 12.5 units/kg of the total body weight of the subject per leg (i.e. 10 to 12.5 units/kg for unilateral administration and 20 to 25 units/kg for bilateral administration.) Other examples include 10 to 12, 10 to 11.5, 10 to 11, and 10 to 10.5 units/kg of the total body weight of the subject per leg. For administration to the gastrocnemius, the dosage may, for example, be 6 to 7.5 units/kg, 6 to 7 units/kg, or 6 to 6.5 units/kg per leg. For administration to the soleus, the dosage may, for example, be 4 to 5 units/kg or 4 to 4.5 units/kg per leg.

Alternatively, a higher dose may be administered if the subject has not exhibited a desired response to previous treatment with botulinum neurotoxin. A subject's response may, for example, be measured using the Physician's Global Assessment (PGA) of response to treatment. The PGA measures response on a scale from −4 to +4, with −4 indicating that response is markedly worse, to 0, which indicates no change, to +4 indicating that response has markedly improved. For example, a desired response may be a PGA score following treatment of 0.25 or above, 0.5 or above, 1 or above, 2 or above, 3 or above, or 4.

An example of such a higher dose is 12.5 to 15 units/kg of the total body weight of the subject per leg (i.e. 12.5 to 15 units/kg for unilateral administration and 25 to 30 units/kg for bilateral administration.) Other examples include 13 to 15, 13.5 to 15, 14 to 15, and 14.5 to 15 units/kg of the total body weight of the subject per leg. For administration to the gastrocnemius, the dosage may, for example, be 7.5 to 9 units/kg per leg. For administration to the soleus, the dosage may, for example, be 5 to 6 units/kg/leg.

In the event of local muscle weakness in a particular muscle, the administration may be to a different muscle. For example, if the subject is suffering from local muscle weakness in a gastrocnemius, administration may be to the subject's other gastrocnemius, or to a soleus, or to some other appropriate muscle. Similarly, if the subject is suffering from local muscle weakness in a soleus, administration may be to the subject's other soleus, or to a gastrocnemius, or to some other appropriate muscle.

Whether administration is to be unilateral or bilateral depends on various factors, including the muscles affected by the patient's spasticity, the severity of the subject's spasticity, the presence of local muscle weakness, and/or the subject's history with treatment with botulinum neurotoxin. For example, administration may be unilateral if only one leg is affected by spasticity and bilateral if both legs are affected. Also, a subject may have spasticity in only one leg but the spasticity may be severe enough that bilateral administration may be appropriate. By contrast, a subject may have spasticity in both legs but local muscle weakness in one leg may mean that unilateral administration to only the other leg is appropriate. Also, unilateral rather than bilateral administration may be appropriate in instances where the subject has had adverse effects resulting from previous treatment with botulinum neurotoxin and bilateral rather than unilateral administration may be appropriate in instances where the subject has not exhibited a desired response following previous unilateral and/or lower dose treatment with botulinum neurotoxin.

In an embodiment, the method of treating a subject for lower limb spasticity comprises: (A) obtaining the weight of the subject; (B) choosing unilateral or bilateral administration; (C) based on the weight of the patient and whether administration is to be unilateral or bilateral, determining the amount of botulinum neurotoxin to administer to the subject; and (D) administering botulinum neurotoxin in such an amount to the subject. In determining the appropriate amount of botulinum neurotoxin, the method may also involve considering whether administration will be to the gastrocnemius and/or to the soleus, the amount of the subject's spasticity, what muscles are affected by the spasticity, the presence of local muscle weakness, the subject's history with treatment with botulinum neurotoxin, the subject's age, the subject's height, the subject's body surface area, and/or the subject's sex. In determining an appropriate dosage amount, adjustments may be made as described above in view of the aforementioned factors.

The present invention also relates in part to a tool for use in determining the amount of an active agent, for example botulinum neurotoxin, to administer to the subject.

In an embodiment, the tool comprises: (A) a first member (1) having thereon a first row of numbers (2) indicative of the weight of the subject and a second row of numbers (3) indicative of a dosage amount of the active agent to be administered to the subject; and (B) a second member (4). The numbers in the second row indicate a specific dosage amount to be administered to a subject having a specific weight and are in longitudinal or radial alignment with corresponding numbers in the first row indicating that specific weight. The first and second members are in a relationship with one another such that one member may be moved by a user to provide an indication as to the dosage amount of the active agent to be administered to a subject having a particular weight.

In some embodiments, both first (1) and second (4) members are planar surfaces and are in longitudinal sliding arrangement with each other. The numbers in the first row (2) are in latitudinal alignment with corresponding numbers in the second row (3). The second member overlays the first and contains an opening (5) that allows for the rows on the first member to be viewed. In certain embodiments, the second member contains two or more openings (6) with each opening allowing for one row on the first member to be viewed. One member may be slid in relation to the other with the numbers viewable through the opening or openings indicating a specific weight and the dosage amount to be administered to a subject having that specific weight. In certain embodiments, the first planar surface folds around and encloses the second planar surface.

In other embodiments, the first member (1) is a planar surface and the second member (4) is a cursor in a longitudinal sliding arrangement with respect to the first member. The numbers in the first row (2) are in latitudinal alignment with corresponding numbers in the second row (3). The cursor may be slid along the first member such that it indicates a specific weight and the dosage amount to be administered to a subject having that specific weight. The indication may be by way of a line (7) on the cursor which overlays a specific weight and the dosage amount to be administered to a subject having that specific weight. In such an embodiment, the cursor may, for example, be transparent or translucent to allow for the user to see the numbers underneath the line. Alternatively, the cursor may contain an opening (5) or openings (6) which allow for the numbers on the first member to be viewable by the user.

In yet other embodiments, the first member (1) is a circular planar surface and the second member (4) is a dial that pivots around the center point of the first member. The numbers in the first row (2) are in radial alignment with corresponding numbers in the second row (3). The cursor may be pivoted such that it indicates a specific weight and the dosage amount to be administered to a subject having that specific weight. The indication may be by way of a line (7) on the cursor which overlays a specific weight and the dosage amount to be administered to a subject having that specific weight. In such an embodiment, the cursor may, for example, be transparent or translucent to allow for the user to see the numbers underneath the line. Alternatively, the cursor may contain an opening (5) or openings (6) which allow for the numbers on the first member to be viewable by the user.

In certain embodiments of the tool, the first member (1) contains, in lieu of or in addition to the aforementioned second row of numbers (3) which indicate a dosage amount of the active agent to be administered to the subject, one or more rows of numbers (8) which indicate the dosage amount of the active agent to be administered to particular muscles of the subject. The numbers in the row or rows which indicate a specific dosage amount to be administered to a muscle of a subject having a specific weight are in longitudinal or radial alignment with corresponding numbers in the first row indicating that specific weight. In embodiments wherein these rows (8) are in addition to the aforementioned second row of numbers (3) which indicate a dosage amount of the active agent to be administered to the subject, the second row of numbers represent the total dosage amount to be administered to the subject.

In an embodiment, one row of numbers indicates the dosage amount of the active agent to be administered to the gastrocnemius of the leg (9) and another row indicates the dosage mount of the active agent to be administered to the soleus of the leg (10).

In embodiments of the tool that contain an opening (5) or two or more openings (6) in the second member, the opening may contain a window made or transparent or translucent material.

In embodiments wherein the second member contains two or more openings (6), each opening may correspond to a specific row on the first member. For example, one opening may allow for the numbers of the first row (2) to be viewed, another opening may allow for the numbers of the second row (3) to be viewed, and third and fourth openings may allow for the numbers of the additional rows (8) to be viewed.

In embodiments wherein the second member (4) contains an opening (5) or openings (6) through which the numbers of the rows on the first member (1) may be viewed or wherein the second member is transparent or translucent, the second member may contain a first marking (11) aligned with the row of numbers which indicate the weight of the subject (2), a second marking (12) aligned with the row of numbers which indicate the dosage amount of the active agent to be administered to the subject (3), and third (13) and fourth markings (14) aligned respectively with the row indicating the dosage amount of the active agent to be administered to the gastrocnemius of the subject's leg (9) and the row indicating the dosage amount of the active agent to be administered to the soleus of the subject's leg (10). The markings may identify each row respectively, for example as: “Total Weight of the Subject”; “Amount of Agent Per Leg to be Administered to Subject”; “Amount of Agent to be Administered Per Leg to the Gastrocnemius of the Subject”, and “amount of Agent to be Administered Per Leg to the Soleus of the Subject.” In embodiments wherein the second member is transparent or translucent, the markings may be on a label (15) to ensure better visibility.

In an embodiment of the aforementioned method of treating lower limb spasticity in a subject, the dosage amount to be administered is determined using a tool as described above.

The invention further relates in part to a composition for use in the treatment of lower limb spasticity in a subject, particularly one which is 18 years of age or under, for example one which is between 2 and 17 years of age. The composition comprises an effective amount of botulinum neurotoxin. In certain embodiments, the composition comprises a diluent, for example a sodium chloride solution. The solution may, for example, be a 0.9% sodium chloride solution.

In certain embodiments, the composition comprises 1 to 1,000 units, 1 to 750 units, 1 to 500 units, 1 to 400 units, 1 to 300 units, or 1 to 200 units of botulin neurotoxin.

In an embodiment, the composition comprises botulinum neurotoxin in the amount of 500 units and 1 to 5 mL of sodium chloride solution, for example 1, 2, 2.5, and 5 mL of the solution. In a particular embodiment, the composition comprises 500 units and 2.5 mL of solution.

In another embodiment, the composition comprises botulinum neurotoxin in the amount of 300 units and 1 to 3 mL of sodium chloride solution, for example 1, 1.5, 2.5, and 3 mL of the solution.

In an embodiment, the composition is formed by reconstituting lyophilized botulinum neurotoxin in a solution, for example the aforementioned sodium chloride solution. Following reconstitution, the composition may be stored under refrigeration at 2 to 8° C. and protected from light.

In an embodiment of the aforementioned method of treating lower limb spasticity in a subject, particularly one which is 18 years of age or under, for example one between 2 and 17 years of age, a composition as described above is administered to the subject. The amount of the composition administered may be the amount determined to be appropriate as described above. For example, for a composition comprising 500 units diluted in 2.5 mL of solution, each 0.1 mL of the composition will contain 20 units. As such, the amount of the composition to be administered to a subject in a unilateral treatment may be 0.05 to 0.075 mL (containing 10 to 15 units) per kg of the total body weight of the subject. Thus, for a subject weighing 50 kg, 2.5 to 3.75 mL of the composition may be administered. The amount of the composition to be administered to a subject in a bilateral treatment may be 0.1 to 0.15 mL (containing 20 to 30 units) per kg of the total body weight of the subject. For a subject weighting 50 kg, 5 to 7.5 mL of the composition may be administered.

In an embodiment, the composition further comprises human serum albumin. For example, 1 to 100 mg/ml, 1 to 50 mg/ml, 1 to 25 mg/ml, or 25 mg/ml.

In an embodiment, the composition further comprises an excipient. The excipient may, for example, be a filler, a binder, a disintegrant, an anti-adherent, a solvent, a buffering agent, a preservative, or a humectant. Examples of fillers include cellulose, lactose, sucrose, glucose, mannose, and sorbitol. The composition may comprise, for example, 10 mg/ml lactose. Examples of binders include gelatin, cellulose, polyvinyl pyrrolidone, starch, and sucrose. Examples of disintegrants include polyvinyl pyrrolidone and carboxymethyl cellulose. Examples of preservatives include parabens. Examples of solvents include water, oils, glycerol, propylene glycol, and ethanol. Examples of buffering agents include phosphates, carbonates, citrates, and lactates. Examples of humectants include glycerol, ethylene glycol, and polyethylene glycol (PEG).

### EXAMPLES

### Example 1

The efficacy of botulinum neurotoxin in treating lower limb spasticity was evaluated in a double-blind, placebo-controlled multicenter study in patients 2 to 17 years of age suffering from spasticity because of cerebral palsy causing dynamic equinus foot deformity.

A total of 235 toxin naïve or non-naïve patients with a Modified Ashworth Score of grade 2 or greater at the ankle plantar flexor were enrolled to receive DYSPORT® or placebo.

Seventy-nine patients received 10 units/kg/leg, 79 patients received 15 units/kg/leg, and 77 patients received placebo. Administration was by injection into the gastrocnemius and soleus muscles. Forty one percent of patients (n=66) were treated bilaterally and received a total DYSPORT® dose of either 20 Units/kg (n=37) or 30 Units/kg (n=29).

TABLE 1

| | Week | Placebo | 10 units/kg/leg | 15 units/kg/leg |
|---|---|---|---|---|
| LS mean change | 4 | −0.5 | −0.9* | −1.0* |
| from baseline in | 12 | −0.5 | −0.8* | −1.0* |
| ankle plantar | | | | |
| flexor muscle | | | | |
| tone on the MAS | | | | |
| LS mean PGA of | 4 | 0.7 | 1.5* | 1.5* |
| response to | 12 | 0.4 | 0.8* | 1.0* |
| treatment | | | | |

LS = least square
*p < 0.05

The patients' change in MAS score following treatment and the PGA score for treatment were measured. As shown above, treatment with botulinum neurotoxin significantly reduced MAS in ankle plantar flexor muscle tone as compared with placebo. Treatment with botulinum neurotoxin also resulted in a significantly greater mean PGA score as compared with placebo.

### Example 2

Five hundred units of lyophilized DYSPORT® are gently mixed with 2.5 mL of preservative-free 0.9% sodium chloride solution in a vial, forming an initial DYSPORT® solution. An additional 2.5 mL of preservative-free 0.9% sodium chloride solution is drawn into a 5 mL syringe. The syringe is then used to draw the aforementioned DYSPORT® solution from the vial without inverting. The resulting solution is mixed gently in the syringe to form a 10 units/0.1 mL injectable solution. The solution is stored in the syringe in a refrigerator at 2 to 8° C. and protected from light. The composition is used within 24 hours and, if not used, is discarded.

### Example 3

Three hundred units of lyophilized DYSPORT® are gently mixed with 1.5 mL of preservative-free 0.9% sodium chloride solution in a vial, forming an initial DYSPORT® solution. An additional 1.5 mL of preservative-free 0.9% sodium chloride solution is drawn into a 3 mL syringe. The syringe is then used to draw the aforementioned DYSPORT® solution from the vial without inverting. The resulting solution is mixed gently in the syringe to form a 10 units/0.1 mL injectable solution. The solution is stored in the syringe in a refrigerator at 2 to 8° C. and protected from light. The composition is used within 24 hours and, if not used, is discarded.

### Example 4

A physician treating a 10-year old patient for severe lower limb spasticity in the left soleus (MAS score of 3) first obtains the weight of the patient. The weight is found to be 30 kg. As the patient is exhibiting spasticity in the soleus of his left leg, the physician determines that administration should be unilateral and to the soleus of the left leg. Consulting a dosing tool, it is determined that a dose of 120 to 180 units is appropriate. The patient's history is then reviewed. As it is found that the patient has had no adverse effects from previous treatment with botulinum neurotoxin and the level of spasticity was severe, it is determined that 180 units are to be administered.

A 10 unit/0.1 mL injectable DYSPORT® solution in a syringe is prepared as described in Example 2. 1.8 mL of the solution is administered by injection into the patient's soleus. The syringe, along with the remainder of the solution therein, is discarded.

The above steps are repeated in 16 weeks.

### Example 5

A physician treating a 12-year old patient for minor lower limb spasticity in both left and right gastrocnemius (MAS score of 1+) first obtains the weight of the patient. The weight is found to be 40 kg. As the patient is exhibiting spasticity in the gastrocnemius of both legs, the physician determines that administration should be bilateral and to the gastrocnemius of both legs. Consulting a dosing tool, it is determined that a dose of 240 to 360 units to each leg is appropriate. The patient's history is then reviewed. As it is found that the patient has had adverse effects from previous treatment with botulinum neurotoxin and the level of spasticity was minor, it is determined that 240 units are to be administered to each leg

A 10 unit/0.1 mL injectable DYSPORT® solution in a syringe is prepared as described in Example 2. 2.4 mL of the solution is administered by injection into the gastrocnemius of each of the patient's legs. The syringe, along with the remainder of the solution therein, is discarded.

The above steps are repeated in 18 weeks.

### Example 6

A physician treating an 8-year old patient for minor lower limb spasticity in the left soleus and the right gastrocnemius (MAS score of 1) first obtains the weight of the patient. The weight is found to be 20 kg. The physician determines that administration should be unilaterally to the soleus of the left leg and unilaterally to the gastrocnemius of the right leg. Consulting a dosing tool, it is determined that a dose of 80 to 120 units to the left soleus and 120 to 180 units to the right gastrocnemius is appropriate. The patient's history is then reviewed. As it is found that the patient has had no adverse effects from previous treatment with botulinum neurotoxin and the level of spasticity was average, it is determined that 100 units are to be administered to the left soleus and 150 units are to be administered to the right gastrocnemius.

A 10 unit/0.1 mL injectable DYSPORT® solution in a syringe is prepared as described in Example 2. One milliliter of the solution is administered by injection into the left soleus and 1.5 mL is administered by injection into the right gastrocnemius.

The above steps are repeated in 17 weeks.

### Example 7

A physician treating an 8-year old patient for minor lower limb spasticity in the left soleus and the right gastrocnemius (MAS score of 1) first obtains the weight of the patient. The weight is found to be 20 kg. The physician determines that administration should be unilaterally to the soleus of the left leg and unilaterally to the gastrocnemius of the right leg. Consulting a dosing tool, it is determined that a dose of 80 to 120 units to the left soleus and 120 to 180 units to the right gastrocnemius is appropriate. The patient's history is then reviewed. The patient exhibited local muscle weakness in the right gastrocnemius. However, the patient has had no adverse effects from previous treatment with botulinum neurotoxin and the level of spasticity was average. It is determined that 100 units are to be administered to the left soleus and, due to muscle weakness, 120 units are to be administered to the right gastrocnemius.

A 10 unit/0.1 mL injectable DYSPORT® solution in a syringe is prepared as described in Example 2. One milliliter of the solution is administered by injection into the left soleus and 1.2 mL is administered by injection into the right gastrocnemius.

The above steps are repeated in 17 weeks.

### Example 8

A physician treating a 12-year old patient for minor lower limb spasticity in both left and right gastrocnemius (MAS score of 1+) first obtains the weight of the patient. The weight is found to be 40 kg. As the patient is exhibiting spasticity in the gastrocnemius of both legs, the physician determines that administration should be bilateral and to the gastrocnemius of both legs. Consulting a dosing tool, it is determined that a dose of 240 to 360 units to each leg is appropriate. The patient's history is then reviewed. It is found that the patient has had no adverse effects from previous treatment with botulinum neurotoxin but that such treatment did not achieve a desired result (PGA was 0). In view of this and that severity of the spasticity was average, it is determined that 360 units are to be administered to each leg

A 10 unit/0.1 mL injectable DYSPORT® solution in a syringe is prepared as described in Example 2. 3.6 mL of the solution is administered by injection into the gastrocnemius of each of the patient's legs. The syringe, along with the remainder of the solution therein, is discarded.

The above steps are repeated in 17 weeks.

### Example 9

A physician treating a 12-year old patient for minor lower limb spasticity in both left and right gastrocnemius (MAS score of 1) first obtains the weight of the patient. The weight is found to be 40 kg. As the patient is exhibiting spasticity in the gastrocnemius of both legs, the physician determines that administration should be bilateral and to the gastrocnemius of both legs. Consulting a dosing tool, it is determined that a dose of 240 to 360 units to each leg is appropriate. The patient's history is then reviewed. It is found that the patient has had no adverse effects from previous treatment with botulinum neurotoxin but that such treatment did not achieve a desired result (PGA was 0.03). In view of this and that severity of the spasticity was average, it is determined that 360 units are to be administered to each leg

A 10 unit/0.1 mL injectable DYSPORT® solution in a syringe is prepared as described in Example 2. 3.6 mL of the solution is administered by injection into the gastrocnemius of each of the patient's legs. The syringe, along with the remainder of the solution therein, is discarded.

The above steps are repeated in 17 weeks.

1. A method for treating lower limb spasticity, the method comprising administering to a subject aged 18 years or younger an effective amount of botulinum neurotoxin.

2. The method of claim 1, wherein the subject is between 2 and 17 years of age.

3. The method of claim 1, wherein the botulinum neurotoxin is of serotypes A, B, C, D, E, F, or G, or a recombinant engineered botulinum neurotoxin having an amino acid sequence that has at least 50% sequence identity to the amino acid sequence of a botulinum neurotoxin of serotypes A, B, C, D, E, F, or G.

4. The method of claim 1, wherein the botulinum neurotoxin is of serotype A.

5. The method of claim 1, wherein the botulinum neurotoxin is administered unilaterally or bilaterally.

6. The method of claim 1, wherein botulinum neurotoxin is administered to a leg of the subject in an amount of 10 to 15 units per kilogram of the total body weight of the subject.

7. The method of claim 1, wherein botulinum neurotoxin is administered to the gastrocnemius and/or soleus of a leg of the subject.

8. The method of claim 1, wherein botulinum neurotoxin is administered in an amount of 6 to 9 units per kilogram of the total body weight of the subject to the gastrocnemius of a leg of the subject and/or in an amount of 4 to 6 units per kilogram of the total body weight of the subject.

9. The method of claim 1, wherein the dosage amount of botulinum neurotoxin to be administered to the subject is determined based on one or more of the following factors: whether administration is unilateral or bilateral; the subject's weight; the amount of spasticity; what muscles are affected by spasticity; whether the subject exhibited adverse effects following previous treatment with botulinum neurotoxin, whether the subject exhibits local muscle weakness; whether the subject has exhibited a desired response to previous treatment with botulinum neurotoxin; the subject's age; the subject's height; the subject's body surface area; and the subject's sex.

10. The method of claim 1, comprising:
(a) obtaining the subject's weight;
(b) choosing unilateral or bilateral administration;
(c) based on the subject's weight and whether unilateral or bilateral administration is chosen, determining the amount of botulinum neurotoxin to administer to the subject; and
(d) administering botulinum neurotoxin in the determined amount to the subject.

11. A tool for use in determining a dosage amount of an active agent to be administered to a subject, the tool comprising:
a first member having thereon a first row of numbers indicative of the weight of the subject and a second row of numbers indicative of a dosage amount of the active agent to be administered to the subject, wherein the numbers in the first row indicating a specific weight are in latitudinal or radial alignment with corresponding numbers in the second row indicating a specific dosage amount to be administered to a subject having that specific weight; and
a second member;
wherein the first and second members are in a relationship with one another such that one member may be moved by a user to provide an indication as to the dosage amount of the active agent to be administered to a subject having a particular weight.

12. The tool of claim 11, wherein the first and second members are both planar surfaces that are in a longitudinal sliding arrangement with each another, the numbers in the first row indicating a specific weight are in latitudinal alignment with corresponding numbers in the second row indicating a specific dosage amount to be administered to a subject having that specific weight, and the second member overlays the first and contains an opening that allows for the rows on the first member to be viewed or which comprises two or more openings with each opening allowing for one row on the first member to be viewed, wherein one member may be slid in relation to the other with the numbers viewable through the opening or openings indicating a specific weight and the dosage amount to be administered to a subject having that specific weight.

13. The tool of claim 11, wherein the first member is a planar surface and the second member is a cursor in a longitudinal sliding arrangement with respect to the first member, the numbers in the first row indicating a specific weight are in latitudinal alignment with corresponding numbers in the second row indicating a specific dosage amount to be administered to a subject having that specific weight, and the cursor may be slid along the first member such that it indicates a specific weight and the dosage amount to be administered to a subject having that specific weight.

14. The tool of claim 11, wherein the first member is a circular planar surface and the second member is a dial that pivots around the center point of the first member, the numbers in the first row indicating a specific weight are in radial alignment with corresponding numbers in the second row indicating a specific dosage amount to be administered to a subject having that specific weight, wherein the cursor may be pivoted such that it indicates a specific weight and the dosage amount to be administered to a subject having that specific weight.

15. The tool of claim 13, wherein: (a) the indication is by way of a line on the cursor which overlays a specific weight and the dosage amount to be administered to a subject having that specific weight; or (b) the indication is by way of an opening in the cursor that allows for the rows on the first member to be viewed or two or more openings with each opening allowing for one row on the first member to be viewed and wherein the numbers viewable through the opening or openings indicate a specific weight and the dosage amount to be administered to a subject having that specific weight.

16. The tool of claim 14, wherein: (a) the indication is by way of a line on the cursor which overlays a specific weight and the dosage amount to be administered to a subject having that specific weight; or (b) the indication is by way of an opening in the cursor that allows for the rows on the first member to be viewed or two or more openings with each opening allowing for one row on the first member to be viewed and wherein the numbers viewable through the opening or openings indicate a specific weight and the dosage amount to be administered to a subject having that specific weight.

17. The tool of claim 11, wherein the first member further has thereon a row of numbers indicative of the dosage amount of the active agent to be administered to a particular muscle of the subject and the numbers in the row which indicate a specific dosage amount to be administered to the muscle of a subject having a specific weight are in longitudinal or radial alignment with corresponding numbers in the first row indicating that specific weight.

18. The tool of claim 11, wherein the first member further has thereon two rows of numbers, each row indicative of the dosage amount of the active agent to be administered to different muscles of the patient and the numbers in the rows which indicate a specific dosage amount to be administered to the muscles of a subject having a specific weight are in longitudinal or radial alignment with corresponding numbers in the first row indicating that specific weight.

19. The tool of claim 11, wherein the second row indicates the total dosage amount of the active agent to be administered to a single leg of the subject and the first member further has thereon two further rows of numbers, one row indicating the dosage amount of the active agent to be administered to the gastrocnemius of the leg and another row indicating the dosage mount of the active agent to be administered to the soleus of the leg, the numbers in the rows which indicate a specific dosage amount to be administered to the muscles of a subject having a specific weight being in longitudinal or radial alignment with corresponding numbers in the first row indicating that specific weight.

20. The method of claim 1, wherein the dosage amount of botulinum neurotoxin to be administered to the subject is determined using a tool comprising:
a first member having thereon a first row of numbers indicative of the weight of the subject and a second row of numbers indicative of a dosage amount of the active agent to be administered to the subject, wherein the numbers in the first row indicating a specific weight are in latitudinal or radial alignment with corresponding numbers in the second row indicating a specific dosage amount to be administered to a subject having that specific weight; and
a second member;
wherein the first and second members are in a relationship with one another such that one member may be moved by a user to provide an indication as to the dosage amount of the active agent to be administered to a subject having a particular weight.
